(19) United States
(12) Patent Application Publication
(10) Pub. No.: US 2015/0320458 A1
(43) Pub. Date: Nov. 12, 2015

(54) SURGICAL INSTRUMENT AND METHOD

(71) Applicant: Warsaw Orthopedic, Inc., Memphis, TN (US)

(72) Inventor: William Alan Rezach, Atoka, TN (US)

(73) Assignee: Warsaw Orthopedic, Inc., Memphis, TN (US)

(21) Appl. No.: 14/271,224

(22) Filed: May 6, 2014

(51) Int. Cl. A61B 17/70

(52) U.S. Cl. CPC A61B 17/7085

(57) ABSTRACT

A surgical instrument includes a first member that defines a first longitudinal axis and includes a first pivot engageable with a first spinal construct connected with a first vertebral surface. A second member includes a second pivot engageable with a second spinal construct connected with a second vertebral surface. The second member is axially translatable relative to the first member along the first longitudinal axis such that the first vertebral surface is moved relative to the second vertebral surface. The first member is engageable to rotate the first spinal construct relative to the first member and the second member is engageable to rotate the second spinal construct relative to the second member. Systems and methods are disclosed.

### TECHNICAL FIELD

The present disclosure generally relates to medical devices for the treatment of spinal disorders, and more particularly to a surgical instrument and method for correction of a spine disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. Correction treatments used for positioning and alignment of vertebrae may employ implants, such as, for example, spinal constructs and interbody devices, for stabilization of a treated section of a spine. In some embodiments, the spinal constructs may be manipulated with surgical instruments for compression and distraction of vertebrae. This disclosure describes an improvement over these prior art technologies.

### SUMMARY

In one embodiment, a surgical instrument is provided. The surgical instrument comprises a first member that defines a first longitudinal axis and includes a first pivot engageable with a first spinal construct connected with a first vertebral surface. A second member includes a second pivot engageable with a second spinal construct connected with a second vertebral surface. The second member is axially translatable relative to the first member along the first longitudinal axis such that the first vertebral surface is moved relative to the second vertebral surface. The first member is engageable to rotate the first spinal construct relative to the first member and the second member is engageable to rotate the second spinal construct relative to the second member In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a spinal correction system in accordance with the principles of the present disclosure;

FIG. 2 is a perspective view of components of the system shown in FIG. 1;

FIG. 3 is a perspective view of components of one embodiment of a spinal correction system in accordance with the principles of the present disclosure disposed with vertebrae; and

FIG. 4 is a perspective view of the components and vertebrae shown in FIG. 3.

### DETAILED DESCRIPTION

The exemplary embodiments of the system and related methods of use disclosed are discussed in terms of medical devices for the treatment of musculoskeletal disorders and more particularly, in terms of a surgical system and a method for correction of a spine disorder. In some embodiments, the present system includes a surgical instrument configured for use with procedures for treating spine trauma. In some embodiments, the surgical instrument can be employed with fixed axis implants and multi-axial implants.

In some embodiments, the present system includes a surgical instrument that can be employed to independently and/or separately provide vertebral body distance, for example, vertebrae position along a spinal rod and sagittal profile, for example, vertebrae angle relative to the spinal rod. In some embodiments, the surgical instrument comprises a lead screw that allows for parallel compression and/or distraction. In some embodiments, the surgical instrument comprises screws that control the pivot angle to control a sagittal angle of vertebrae to a spinal construct. In some embodiments, the surgical instrument can be employed as a posterior trauma instrument. In one embodiment the lead screw would be replaced with a pivot joint such that the lead screw is replaced with ‘scissor style’ handles.

In one embodiment, the surgical instrument can compress and/or distract vertebrae and restore curvature of a spine. In one embodiment, the surgical instrument can lock sagittal alignment of vertebrae, In one embodiment, the surgical instrument includes a four bar linkage to manipulate a pivot angle of a screw.

In one embodiment, the present system can be employed with a method that includes the steps of connecting the instrument to screws attached to vertebrae. In one embodiment, the method that includes the steps of distracting the vertebrae while maintaining a selected angle of vertebrae; and correcting a sagittal plane of the vertebrae after distracton. In one embodiment, the present system can be employed with a method that includes the steps of correcting an angle of the vertebrae in a sagittal plane and distracting the vertebrae while maintaining the sagittal plane correction.

In some embodiments, one or all of the components of the system may be disposable, peel pack and/or pre packed sterile devices. One or all of the components of the system may be reusable. The system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value, When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

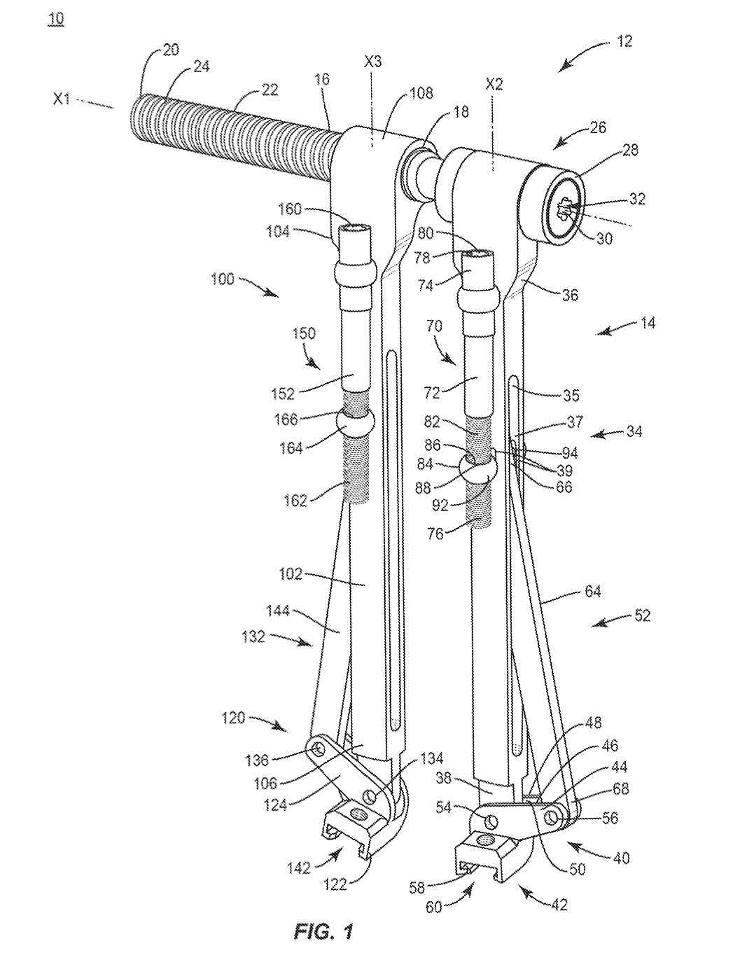

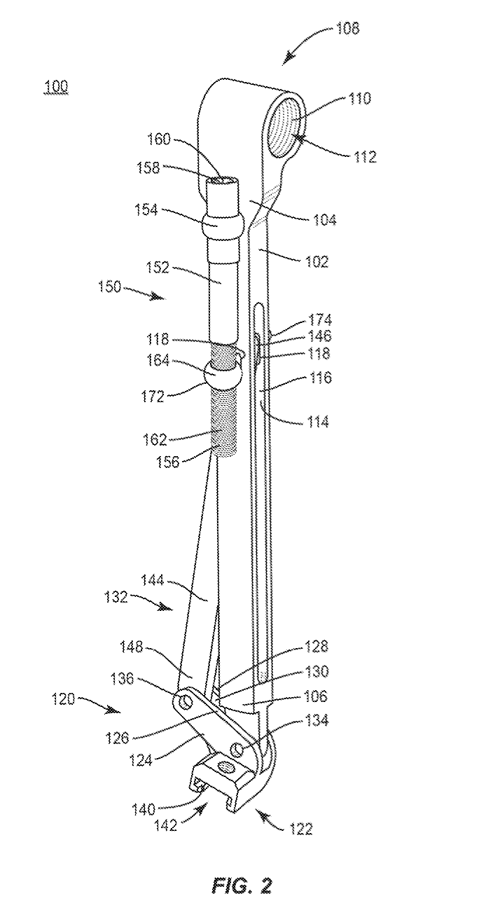

The following discussion includes a description of a system in accordance with the principles of the present disclosure. Alternate embodiments are also disclosed. Reference is made in detail to the exemplary embodiments of the present disclosure, which are illustrated in the accompanying figures. Turning to FIGS. 1 and 2, there are illustrated components of a system, such as, for example, a spinal correction system 10.

The components of system 10 can be fabricated from biologically acceptable materials suitable for medical applications, including metals, synthetic polymers, ceramics and/or their composites. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials The components of system 10 may be monolithically formed, integrally connected or include fastening elements and/or instruments, as described herein.

System 10 includes a surgical instrument 12 configured for engagement with spinal constructs to correct a spinal disorder, such as, for example, various deformities including trauma and/or fracture of vertebrae, which may include a sagittal deformity, as described herein. Instrument 12 includes a member 14. Member 14 includes a shaft 16. Shaft 16 extends between an end 18 and an end 20 and defines a longitudinal axis X1. Shaft 16 has a cylindrical cross section configuration. In some embodiments, shaft 16 may have alternate cross section configurations, such as, for example, oval, oblong, triangular, rectangular, square, polygonal, irregular, uniform, non-uniform, variable and/or tapered.

Shaft 16 includes an outer surface 22. A portion of surface 22 includes a rack 24 configured for engagement with a member 100, as discussed herein. Rack 24 is configured to facilitate relative axial translation of members 14, 100 and/or selective compression and/or distraction of vertebrae, as discussed herein. Rack 24 includes an external thread form that is engageable with a member 100, as described herein. In some embodiments, the external thread form may include a single thread turn or a plurality of discrete threads. In some embodiments, all or only a portion of surface 22 may include a gear rack and/or teeth engageable with a gear of member 100 to facilitate relative axial translation of members 14, 100 and/or selective compression and/or distraction of vertebrae, as discussed herein.

Member 14 includes an actuator 26 disposed at end 18 of shaft 16. Actuator 26 is configured to facilitate relative axial translation of members 14, 100, as discussed herein. Actuator 26 includes an end 28 configured for engagement with a surgical tool. End 28 includes an inner surface 30 that defines a cavity 32. In one embodiment, cavity 32 includes a hexagonal cross section configuration. Engagement of the surgical tool with actuator 26 causes rotation of actuator 26 and relative axial translation of members 14, 100, as described herein.

Member 14 includes an arm 34. Arm 34 extends between an end 36 and an end 38. Arm 34 defines a longitudinal axis X2 that extends transverse to axis X1. Arm 34 includes an inner surface 35 that defines a cavity, such as, for example, a passageway 37 configured for disposal of a linkage 52, as described herein. Surface 35 defines slots 39 configured for moveable disposal of an actuator 70, as described herein.

End 38 includes a pivot, which comprises a foot 40 configured for engagement with a spinal construct connected with a vertebral surface, as described herein. Foot 40 includes an angled configuration to facilitate rotation of a spinal construct. Foot 40 includes a section, such as, for example, a capture element 42 connected to a section 44. Foot 40 includes spaced apart walls 46, 48 that define a cavity, such as, for example, a channel 50. Channel 50 is configured for disposal of arm 34 and linkage 52, described herein. Foot 40 is attached to arm 34 and linkage 52 via a screw, post and/or pins 54, 56. Foot 40 is movably connected to arm 34 via a pivot pin 54 to facilitate pivotal movement of foot 40 and rotation thereof relative to axis X2.

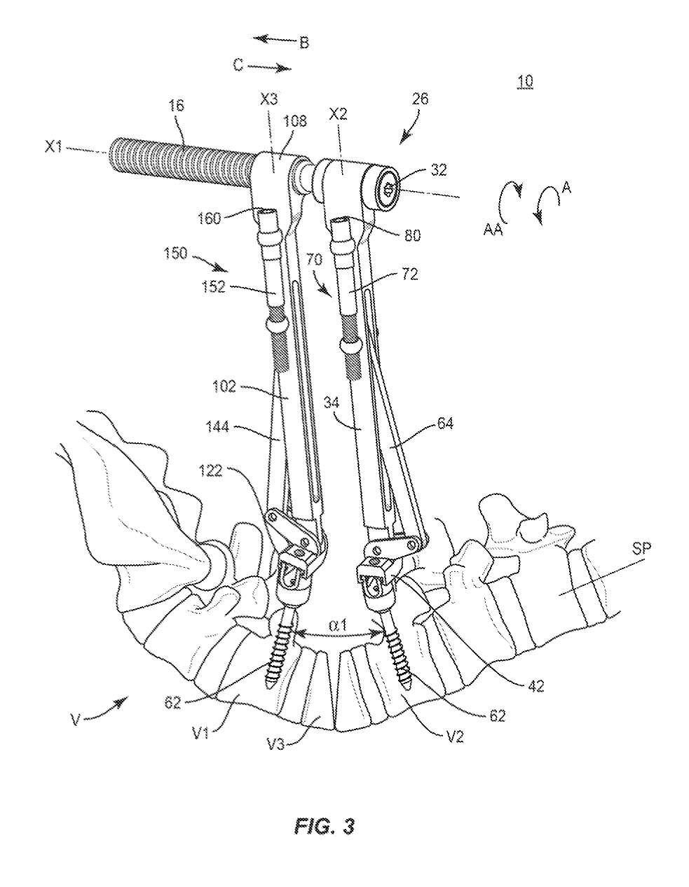

Capture element 42 includes an inner surface 58 that defines a cavity 60. Cavity 60 is configured for disposal of a proximal end of a spinal construct, such as, for example, a bone screw 62, as shown in FIG. 3 and described herein, to facilitate rotation of bone screw 62 relative to and about pivot pin 54. In some embodiments, capture element 42 may be disposed in alternate orientations relative to section 44, such as, for example, perpendicular, transverse and/or other angular orientations such as acute or obtuse, co-axial and/or may be offset or staggered. In some embodiments, the spinal construct may include fasteners, plates, connectors and/or spinal rods.

Linkage 52 is disposed with arm 34 and is configured for rotating bone screw 62 disposed with foot 40, as described herein. Linkage 52 includes a link 64 that is disposed in a transverse orientation relative to axis X2. In some embodiments, link 64 may be disposed in alternate orientations relative to axis X2, such as, for example, parallel and/or other angular orientations such as acute or obtuse, co-axial and/or may be offset or staggered. Link 64 extends between an end 66 and an end 68. End 66 is connected to actuator 70, as described herein. End 68 is connected to section 44 via link 56 to cause movement thereof and rotation of bone screw 62 relative to and about pivot pin 54.

Actuator 70 includes a shaft 72. Shaft 72 is disposed with arm 34 along axis X2. Shaft 72 extends between an end 74 and an end 76. End 74 is configured for engagement with a surgical tool. End 74 includes an inner surface 78 that defines a cavity 80. In one embodiment, cavity 80 includes a hexagonal cross section configuration. End 74 is fixed with arm 34. A portion of end 76 includes a threaded surface 82 configured to facilitate axial translation of a ring 84 relative to shaft 72, as described herein.

Actuator 70 includes ring 84, which includes an inner surface 86 that defines a cavity 88. Cavity 88 is configured for disposal of shaft 72. Surface 86 includes a threaded surface, not shown, that engages surface 82 to facilitate axial translation of ring 84 relative to shaft 72. Ring 84 includes an outer surface 92. A pin 94 extends from surface 92 for disposal with slots 39 and connection to link 64. Engagement of the surgical tool with actuator 70 causes rotation of shaft 72 and linear translation of ring 84 along shaft 72 such that pin 94 translates within slots 39. Pin 94 is connected to link 64 such that translation of pin 94 causes link 64 to translate for rotating foot 40 and bone screw 62 relative to and about pivot pin 54, as described herein.

Member 100 includes an arm 102. Arm 102 extends between an end 104 and an end 106. Arm 102 defines a longitudinal axis X3 that extends transverse to axis X1 and parallel to axis X2. Arm 102 includes an engagement portion 108 disposed at end 104 configured for engagement with member 14. Portion 108 includes an inner surface 110 that defines a cavity, such as, for example, a passageway 112, as shown in FIG. 2. Passageway 112 extends along axis X1. Passageway 112 is configured for moveable disposal of shaft 16. Surface 110 includes a threaded surface configured for engagement with rack 24 such that member 100 is axially translatable relative to member 14 along axis X1 to move a first vertebral surface relative to a second vertebral surface, as described herein.

Arm 102 includes an inner surface 114 that defines a cavity, such as, for example, a passageway 116 configured for disposal of a linkage 132, as described herein. Surface 114 defines slots 118 configured for moveable disposal of an actuator 150, as described herein.

End 106 includes a pivot, which comprises a foot 120 configured for engagement with a spinal construct connected with a vertebral surface, as described herein. Foot 120 includes an angled configuration to facilitate rotation of a spinal construct. Foot 120 includes a section, such as, for example, a capture element 122 connected to a section 124. Foot 120 includes spaced apart walls 126, 128 that define a cavity, such as, for example, a channel 130. Channel 130 is configured for disposal of arm 102 and linkage 132, as described herein. Foot 120 is attached to arm 102 and linkage 132 via a screw, post and/or pins 134, 136. Foot 120 is movably connected to arm 102 via a pivot pin 134 to facilitate pivotal movement of foot 120 and rotation thereof relative to axis X3.

Capture element 122 includes an inner surface 140 that defines a cavity 142. Cavity 142 is configured for disposal of a proximal end of a spinal construct, such as, for example, a bone screw 62, as described herein, to facilitate rotation of bone screw 62 relative to and about pin 134. In some embodiments, capture element 122 may be disposed in alternate orientations relative to section 124, such as, for example, perpendicular, transverse and/or other angular orientations such as acute or obtuse, co-axial and/or may be offset or staggered.

Linkage 132 is disposed with arm 102 and is configured for rotating bone screw 62 disposed with foot 120, as described herein. Linkage 132 includes a link 144 that is disposed in a transverse orientation relative to axis X3. In some embodiments, link 144 may be disposed in alternate orientations relative to axis X3, such as, for example, parallel and/or other angular orientations such as acute or obtuse, co-axial and/or may be offset or staggered. Link 144 extends between an end 146 and an end 148. End 146 is connected to actuator 150, as described herein. End 148 is connected to section 124 via link 144 to cause movement thereof and rotation of bone screw 62 relative to and about pivot pin 134. In some embodiments, linkage 52 and/or linkage 132 may comprise a four bar linkage with the members.

Actuator 150 includes a shaft 152. Shaft 152 is disposed with arm 102 along axis X3. Shaft 152 extends between an end 154 and an end 156. End 154 is configured for engagement with a surgical tool. End 154 includes an inner surface 158 that defines a cavity 160. In one embodiment, cavity 160 includes a hexagonal cross section configuration. End 154 is fixed with arm 102. A portion of end 156 includes a threaded surface 162 configured to facilitate axial translation of a ring 164 relative to shaft 152, as described herein.

Actuator 150 includes ring 164, which includes an inner surface 166 that defines a cavity 168. Cavity 168 is configured for disposal of shaft 152. Surface 166 includes a threaded surface, not shown, to engage surface 162 to facilitate axial translation of ring 164 relative to shaft 152. Ring 164 includes an outer surface 172. A pin 174 extends from surface 172 for disposal with slots 118 and connection to link 144. Engagement of the surgical tool with actuator 150 causes rotation of shaft 152 and linear translation of ring 164 along shaft 152 such that pin 174 translates within slots 118. Pin 174 is connected to link 144 such that translation of pin 174 causes link 144 to translate for rotating foot 120 and bone screw 62 relative to and about pivot pin 174, as described herein.

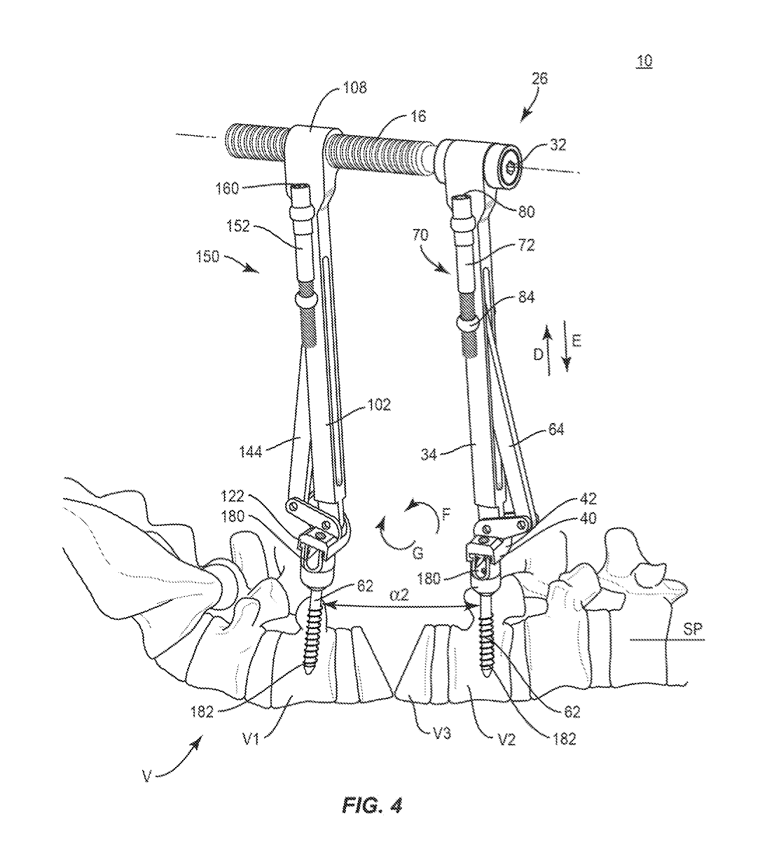

System 10 includes a spinal construct, such as, for example, bone screw 62, as shown in FIGS. 3 and 4. Bone screw 62 includes a posterior end, such as, for example, a head 180 configured for attachment with capture elements 42, 122, and an anterior end, such as, for example, an elongated shaft 182 configured for penetrating tissue. Shaft 182 has a cylindrical cross section configuration and includes an outer surface having an external thread form, In one embodiment, the thread form may include a single thread turn or a plurality of discrete threads. In some embodiments, other engaging structures may be disposed on shaft 182, such as, for example, a nail configuration, barbs, expanding elements, raised elements and/or spikes to facilitate engagement of shaft 182 with tissue, such as, for example, vertebrae.

In assembly, operation and use, as shown in FIGS. 3 and 4, spinal correction system 10, similar to the systems and methods described above, is employed with a surgical procedure, such as, for example, a correction treatment to treat trauma of the spine, such as, for example, thoracolumbar and lumbar fractures. In some embodiments, one or all of the components of system 10 can be delivered or implanted as a pre-assembled device or can be assembled in situ. System 10 may be completely or partially revised, removed or replaced.

For example, system 10 can be employed with a surgical correction treatment of an applicable condition or injury, such as, for example, a trauma of an affected section of a spinal column and adjacent areas within a body, such as, for example, a fractured vertebra V3 of vertebrae V. In some embodiments, system 10 may be employed with one or a plurality of vertebra.

A medical practitioner obtains access to a surgical site including vertebrae V in any appropriate manner, such as through incision and retraction of tissues. Once access to the surgical site is obtained, the particular surgical procedure can be performed for treating a trauma, such as, for example, a spinal fracture.

An incision is made in the body of the patient and a cutting instrument (not shown) creates a surgical pathway for implantation of components of system 10. A preparation instrument (not shown) can be employed to prepare tissue surfaces of vertebrae V, as well as for aspiration and irrigation of a surgical region.

Pilot holes or the like are made in selected vertebra V1 and V2 of vertebrae V adjacent fractured vertebra V3 for receiving bone screws 62, with fractured vertebra V3 being disposed between vertebrae V1, V2. A driver (not shown) is disposed adjacent vertebrae V at a surgical site and is manipulated to drive, torque, insert or otherwise connect bone screws 62 adjacent vertebrae V1 and V2.

Surgical instrument 12 is disposed adjacent a surgical site and manipulated for engagement with bone screws 62 such that vertebrae V can be axially distracted to treat trauma to vertebrae V. Heads 180 are engaged with capture elements 42, 122. Shaft 16 is disposed with passageway 112 of portion 108 such that member 100 can be axially translated relative to member 14 so that vertebrae V can be compressed and/or distracted.

In some embodiments, a surgical driver is disposed with cavity 32 for mating engagement with actuator 26. In some embodiments, the surgical driver is rotated, in the direction shown by arrow A in FIG. 3, to rotate threaded shaft 16 such that member 100 axially translates relative to member 14 and along axis X1, in the direction shown by arrow B. Translation of member 100 relative to member 14, in the direction shown by arrow B, causes distraction of vertebrae V such that vertebra V1 is spaced from vertebra V2 by moving vertebra V2 relative to vertebra V1 In some embodiments, the surgical driver is rotated, in the direction shown by arrow AA, to rotate threaded shaft 16 such that member 100 axially translates relative to member 14 and along axis X1, in the direction shown by arrow C. Translation of member 100 relative to member 14, in the direction shown by arrow C, causes compression of vertebrae V such that vertebra V1 is drawn closer to vertebra. V2 by moving vertebra V2 relative to vertebra V1.

In some embodiments, a surgical driver is disposed with cavity 80 for mating engagement with actuator 70. The surgical driver rotates shaft 72 causing ring 84 to translate along axis X2, as described herein. Pin 94 translates along slots 39, in either of the directions shown by arrows D and E in FIG. 4. Engagement of the surgical driver with actuator 70 causes rotation of shaft 72 and axial translation of ring 84 along shaft 72. Pin 94 is connected to link 64 such that translation of pin 94 causes link 64 to translate for rotating foot 40 and bone screw 62 relative to and about pivot pin 54.

In some embodiments, the surgical driver is manipulated to selectively rotate shaft 72 in a clockwise direction such that pin 94 translates, in the direction shown by arrow D, and foot 40 is rotated, in the direction shown by arrow F. As such, foot 40 selectively rotates bone screw 62, in the direction shown by arrow F, relative to and about pivot pin 54 within a sagittal plane SP of vertebrae V. With bone screw 62 fastened with vertebra V2, vertebra V2 is rotated, in the direction shown by arrow F, relative to and about pivot pin 54 within plane SP. In some embodiments, the surgical driver is manipulated to selectively rotate shaft 72 in a counter-clockwise direction such that pin 94 translates, in the direction shown by arrow E, and foot 40 is rotated, in the direction shown by arrow G. As such, foot 40 selectively rotates bone screw 62, in the direction shown by arrow G, relative to and about pivot pin 54 within plane SR With bone screw 62 fastened with vertebra V2, vertebra V2 is rotated, in the direction shown by arrow G, relative to and about pivot pin 54 within plane SP.

In some embodiments, a surgical driver is disposed with cavity 160 for mating engagement with actuator 150. The surgical driver rotates shaft 152 causing ring 164 to translate along axis X3, as described herein. Pin 174 translates along slots 118, in either of the directions shown by arrows D and E. Engagement of the surgical driver with actuator 150 causes rotation of shaft 152 and axial translation of ring 164 along shaft 152. Pin 174 is connected to link 144 such that translation of pin 174 causes link 144 to translate for rotating foot 120 and bone screw 62 relative to and about pivot pin 136.

In some embodiments, the surgical driver is manipulated to selectively rotate shaft 152 in a clockwise direction such that pin 174 translates, in the direction shown by arrow D, and foot 120 is rotated, in the direction shown by arrow G. As such, foot 120 selectively rotates bone screw 62, in the direction shown by arrow G, relative to and about pivot pin 136 within plane SP. With bone screw 62 fastened with vertebra V1, vertebra V1 is rotated, in the direction shown by arrow G, relative to and about pivot pin 136 within plane SP. In some embodiments, the surgical driver is manipulated to selectively rotate shaft 152 in a counter-clockwise direction such that pin 136 translates, in the direction shown by arrow E, and foot 120 is rotated, in the direction shown by arrow F. As such, foot 120 selectively rotates bone screw 62, in the direction shown by arrow F, relative to and about pivot pin 136 within plane SP. With bone screw 62 fastened with vertebra V1, vertebra V1 is rotated, in the direction shown by arrow F, relative to and about pivot pin 136 within plane SP.

In some embodiments, heads 180 are engaged with capture elements 42, 122 such that shafts 182 are disposed at an angle α1, as shown in FIG. 3, based on an orientation of vertebra V1 relative to vertebra V2 adjacent fractured vertebra V3. In some embodiments, angle α1 is measured between the longitudinal axes of shafts 182.

In one embodiment, member 100 is translated relative to member 14, in the direction shown by arrow B and described herein, initially causing distraction of vertebrae V such that vertebra V1 is spaced from vertebra V2 while maintaining a selected angle of vertebrae V. Thereafter, as shown in FIG. 4, foot 40 selectively rotates bone screw 62 fastened with vertebra V2 to rotate vertebra V2 relative to and about pivot pin 54 within plane SP, as described herein, and foot 102 selectively rotates bone screw 62 fastened with vertebra V1 to rotate vertebra V1 relative to and about pivot pin 136 within plane SP, as described herein. As such, shafts 182 are disposed at an angle α2, as shown in FIG. 4, for treating fractured vertebra V3 and correcting plane SP of vertebrae V.

In one embodiment, foot 40 selectively rotates bone screw 62 fastened with vertebra V2 to rotate vertebra V2 relative to and about pivot pin 54 within plane SP, as described herein, and foot 102 selectively rotates bone screw 62 fastened with vertebra V1 to rotate vertebra. V1 relative to and about pivot pin 136 within plane SP, as described herein. As such, shafts 132 are disposed at an angle α2, as shown in FIG. 3, for initially correcting plane SP of vertebrae V. Thereafter, member 100 is translated relative to member 14, in the direction shown by arrow B and described herein, causing distraction of vertebrae V such that vertebra V1 is spaced from vertebra V2 for treating fractured vertebra V3 while maintaining plane correction of plane SP of vertebrae V.

In some embodiments, surgical instrument 12 compresses and/or distracts vertebra V to restore vertebral body height and restores curvature of vertebrae V by rotating vertebra about a center of rotation corresponding to a bone fastener adjacent a facet joint. In some embodiments, alignment along sagittal plane SP is altered prior to distraction. In some embodiments, a spinal rod may be attached with bone screws 62.

Upon completion of a procedure, as described herein, the surgical instruments, assemblies and non-implanted components of spinal correction system 10 are removed and the incision(s) are closed. One or more of the components of spinal correction system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal correction system 10. In some embodiments, spinal correction system 10 may include one or a plurality of rods, plates, connectors and/or bone fasteners for use with a single vertebral level or a plurality of vertebral levels.

In some embodiments, one or more of bone screws 62 may be engaged with tissue in various orientations, such as, for example, series, parallel, offset, staggered and/or alternate vertebral levels. In some embodiments, one or more of bone screws 62 may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, clips, snaps, friction fittings, compressive fittings, expanding rivets, staples, nails, adhesives, posts, fixation plates and/or posts.

In one embodiment, spinal correction system 10 includes an agent, which may be disposed, packed, coated or layered within, on or about the components and/or surfaces of spinal correction system 10. In some embodiments, the agent may include bone growth promoting material, such as, for example, bone graft to enhance fixation of the components and/or surfaces of spinal correction system 10 with vertebrae. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments, Those skilled in the art will envision other modifications within the scope and spirit of the claims appended hereto.

What is claimed is:

1. A surgical instrument comprising:
a first member defining a first longitudinal axis and including a first pivot engageable with a first spinal construct connected with a first vertebral surface; and
a second member including a second pivot engageable with a second spinal construct connected with a second vertebral surface, the second member being translatable relative to the first member along the first longitudinal axis such that the first vertebral surface is moved relative to the second vertebral surface,
wherein the first member is engageable to rotate the first spinal construct relative to the first member and the second member is engageable to rotate the second spinal construct relative to the second member.

2. A surgical instrument as recited in claim 1 wherein the first member includes an outer surface having a gear rack engageable with the second member.

3. A surgical instrument as recited in claim 1, wherein the first member includes an actuator that facilitates relative axial translation of the members.

4. A surgical instrument as recited in claim 3, wherein the actuator is engageable with a surgical tool to rotate the first member to facilitate relative axial translation of the members.

5. A surgical instrument as recited in claim 1, wherein the first member includes an arm that defines a second longitudinal axis disposed transverse to the first longitudinal axis, the arm including the first pivot.

6. A surgical instrument as recited in claim 1, wherein the first member includes a shaft that defines the first longitudinal axis and an arm that defines a second longitudinal axis disposed transverse to the first longitudinal axis, the arm including the first pivot.

7. A surgical instrument as recited in claim 1, wherein the first member includes an actuator connected with the first pivot for rotating the first pivot.

8. A surgical instrument as recited in claim 7, wherein the actuator is engageable with a surgical tool to rotate the first pivot.

9. A surgical instrument as recited in claim 1, wherein the first member includes a linkage for rotating the first pivot.

10. A surgical instrument as recited in claim 9, wherein the first member includes an actuator connected with the linkage such that the actuator linearly translates to rotate the first pivot.

11. A surgical instrument as recited in claim 1, wherein the first member includes a linkage for rotating the first pivot and the second member includes a linkage for rotating the second pivot.

12. A surgical instrument as recited in claim 11, wherein the members each include an actuator connected with the respective linkage such that the actuators linearly translate to rotate the pivots.

13. A surgical instrument as recited in claim 1, wherein the spinal constructs and the vertebral surfaces are rotated in a sagittal plane of a body.

14. A surgical instrument as recited in claim 1, wherein the first pivot includes a capture element engageable with the first spinal construct.

15. A surgical instrument as recited in Claim wherein the pivots each include a capture element engageable with the respective spinal construct.

16. A surgical instrument comprising:
a first member including a shaft defining a first longitudinal axis and an arm defining a second longitudinal axis disposed transverse to the first longitudinal axis, the arm including an actuator and a linkage connected to a first pivot engageable with a first bone fastener connected with a first vertebral surface; and
a second member including an actuator and a linkage connected to a second pivot engageable with a second bone fastener connected with a second vertebral surface, the second member being axially translatable relative to the shaft along the first longitudinal axis such that the first vertebral surface is moved relative to the second vertebral surface,
wherein the actuator of the first arm linearly translates such that the linkage of the first arm rotates the first bone fastener and the actuator of the second member linearly translates such that the linkage of the second member rotates the second bone fastener.

17. A surgical instrument as recited in claim 16 wherein the bone fasteners and the vertebral surfaces are rotated in a sagittal plane of a body.

18. A method for treating a spine the method comprising the steps of
providing a surgical instrument including a first member including a first pivot, and a second member including a second pivot, the second member being axially translatable relative to the first member, each of the pivots being rotatable relative to the respective member;
providing a first spinal construct disposed with a first vertebral surface of a body;
providing a second spinal construct disposed with a second vertebral surface of the body that is spaced apart from the first vertebral surface;
connecting the first pivot with the first spinal construct and the second pivot with the second spinal construct; and
actuating the members to move the second vertebral surface relative to the first vertebral surface.

19. A method as recited in claim 18, wherein the step of actuating includes axially translating the second member relative to the first member to distract the vertebral surfaces; and subsequently rotating the spinal constructs and the vertebral surfaces in a sagittal plane of a body.

20. A method as recited in claim 18, wherein the step of actuating includes rotating the spinal constructs and the vertebral surfaces in a sagittal plane of a body; and subsequently axially translating the second member relative to the first member to distract the vertebral surfaces.
